(19) United States
(12) Patent Application Publication
(10) Pub. No.: US 2014/0039063 A1
(43) Pub. Date: Feb. 6, 2014

(54) LEUKOTRIENES AND ASTHMA EXACERBATION RISK

(71) Applicant: Nathan Rabinovitch, Denver, CO (US)

(72) Inventor: Nathan Rabinovitch, Denver, CO (US)

(73) Assignee: NATIONAL JEWISH HEALTH, Denver, CO (US)

(21) Appl. No.: 13/978,182

(22) PCT Filed: Jan. 4, 2012
(86) PCT No.: PCT/US12/20186

§ 371 Date: Sep. 18, 2013

Related U.S. Application Data
(60) Provisional application No. 61/429,593, filed on Jan. 4, 2011.

(51) Int. Cl. G01N 33/92; A61K 31/137; G01N 33/53

(52) U.S. Cl. CPC G01N 33/92; G01N 33/5308; A61K 31/137
USPC 514/653; 436/501; 436/71

(57) ABSTRACT

The invention provides methods for predicting the risk for a subject of exacerbation of an inflammatory disease of the airways upon exposure to an environmental agent as well as methods for reducing a subject's risk of exacerbation of an inflammatory disease of the airways upon exposure to an environmental agent.

### CROSS REFERENCE TO RELATED APPLICATIONS

The present application claims the benefit of priority under 35 U.S.C. 119(e) from Provisional Patent Application Ser. No. 61/429,593, filed on Jan. 4, 2011. Provisional Patent Application Ser. No. 61/429,593 is incorporated herein by reference in its entirety.

### GOVERNMENT SUPPORT

This invention was made with government support under grant number ES015510-01, awarded by the National Institutes of Health (NIH/NIEHS). The Government of the United States has certain rights in this invention.

### FIELD OF INVENTION

The present invention is directed toward novel methods of predicting and reducing an individual's risk of exacerbation of an inflammatory disease of the airways upon exposure to an environmental agent.

### BACKGROUND

Leukotrienes are a family of lipid mediators derived from arachidonic acid (ARA) through the 5-lipoxygenase pathway. They are produced by various leukocytes, hence the first part of their name (leuko-). The tri-ene part of the name refers to the number (three) of conjugated double bonds (alkenes). Examples of leukotrienes are LTA₄, LTB₄, LTC₄, LTD₄, LTE₄, and LTF₄, with LTC₄, LTD₄ and LTE₄ often called cysteinyl leukotrienes (CysLTs) due to the presence of the amino acid in their structure. The first leukotriene to be synthesized, leukotriene A₄ (LTA₄), is formed through conversion of ARA located in membrane phospholipids to 5-hydroperoxyeicosatetraenoic (5-HPETE) and LTA₄ through membrane-bound 5-lipoxygenase and 5-lipoxygenase-activating protein (FLAP). In human mast cells, basophils, eosinophils, and macrophages, LTA₄ converts quickly to either LTB₄ (through LTA hydrolase) or LTC₄ by LTC₄ synthase with the incorporation of glutathione (γ-glutamyl-cysteinyl-glycine). LTC₄ is subsequently converted to LTD₄ and then to the stable end product LTE₄ (Rabinovitch, N. Urinary Leukotriene E4. Immunol. Allergy Clin. N. Am. 27:651-664, 2007 and Busse W, Kraft M. Cysteinyl leukotrienes in allergic inflammation: strategic target for therapy. Chest 2005; 127:1312-26).

The CysLTs are important mediators of inflammatory reactions and exert powerful effects on vasoconstriction and bronchoconstriction (Doucet M Y, Jones T R, Ford-Hutchinson A W. Responses of equine trachealis and lung parenchyma to methacholine, histamine, serotonin, prostanoids, and leukotrienes in vitro. Can J Physiol Pharmacol 1990; 68:379-83; Gyllfors P, Kumlin M, Dahlén S E, Gaber F, Ehrs P O, Dahlen B. Relation between bronchial responsiveness to inhaled leukotriene D4 and markers of leukotriene biosynthesis. Thorax 2005 November; 60(11):902-8). CysLTs are thus mediators of inflammatory airway diseases such as asthma and chronic obstructive pulmonary disease (Busse W, Kraft M. Cysteinyl leukotrienes in allergic inflammation: strategic target for therapy. Chest 2005; 127:1312-26; Kanwar S, Johnston B, Kubes P. Leukotriene C4/D4 induces P-selectin and sialyl Lewis (x)-dependent alterations in leukocyte kinetics in vivo. Circ Res 1995; 77:879-887). In particular, CysLTs have been implicated in asthma worsening triggered by exposure to tobacco smoke.

Clinical studies have demonstrated that leukotriene receptor antagonists (LTRAs) are able to reduce rescue treatment requirements, improve pulmonary function and reduce symptoms in adults and children with asthma (Barnes N, Thomas M, Price D, Tate H. The National Montelukast Survey. J Allergy Clin Immunology 2005; 115; 47-54; Becker A, Swern A, Tozzi C A, Yu Q, Reiss T, Knorr B. Montelukast in asthmatic patients 6 years-14 years old with an FEV1>75%. Curr Med Res Opin 2004 October; 20 (10): 1651-9). This relationship between CysLTs and asthma severity appears to not be homogenous across populations. For example, a number of studies have reported that female schoolchildren respond more favorably to the LTRA montelukast than boys (Szefler S J, Phillips B R, Martinez F D, Chinchilli V M, Lemanske R F, Strunk R C, et al. Characterization of within-subject responses to fluticasone and montelukast in childhood asthma. J Allergy Clin Immunol 2005; 115:233-42; Johnston N R, Mandhane P J, Dai J, Duncan J M, Greene J M, Lambert K, et al. Attenuation of the September epidemic of asthma exacerbations in children: a randomized, controlled trial of montelukast added to usual therapy. Pediatrics 2007 September; 120(3): 702-12) and that smoking adults show a greater response than non-smokers (Lazarus S C, Chinchilli V M, Rollings N J, Boushey H A, Chemiack R, Craig T J, et al. Smoking affects response to inhaled corticosteroids or leukotriene receptor antagonists in asthma. Am J Respir Crit. Care Med 2007 Apr. 15; 175 (8): 783-90). Other studies have reported both that urinary LTE₄ (uLTE₄) excretion increases acutely after tobacco smoking (Fauler J, and Frolich J C. Cigarette smoking stimulates cysteinyl leukotriene production in man. Eur J Clin Invest 1997; 27:43-47) and that levels of fractional exhaled nitric oxide (FENO) are lower in schoolchildren with chronic environmental tobacco exposure (Warke T J, Mairs V, Fitch P S, Ennis M, Shields M D. Possible association between passive smoking and lower exhaled nitric oxide in asthmatic children. Arch Environ Health 2003 October; 58(10): 613-6; and Nordvall S L, Janson C, Kalm-Stephens P, Foucard T, Torén K, Alving K. Exhaled nitric oxide in a population-based study of asthma and allergy in schoolchildren. Allergy 2005 April; 60(4): 469-75.). Why antileukotriene medications are effective in some subjects and ineffective in others remains unclear. Some studies suggest that susceptibility to LTRAs is related to differences in CysLT levels between individuals (Rabinovitch N, et al.; Childhood Asthma Research and Education Network of the National Heart, Lung, and Blood Institut, Urinary leukotriene E4/exhaled nitric oxide ratio and montelukast response in childhood asthma. J Allergy Clin Immunol. 2010 September; 126(3):545-51.e1-4. Erratum in: J Allergy Clin Immunol. 2010 November; 126(5):959-61; Szefler S J, et al. Characterization of within-subject responses to fluticasone and montelukast in childhood asthma. J Allergy Clin Immunol 2005; 115:233-42; Cai C, et al. Relationship between urinary cysteinyl leukotriene E4 levels and clinical response to antileukotriene treatment in patients with asthma. Lung 2007 March-April; 185(2): 105-12) while other studies have not observed this relationship (Dahlén S E, et al. Improvement of aspirin-intolerant asthma by montelukast, a leukotriene antagonist. A randomized, double-blind, placebo-controlled trial. Am J Respir Crit. Care Med 2002; 165: 9-14). As such, it is unclear whether susceptibility is primarily a function of increased CysLT production, differences in pharmacokinetic metabolism of the medication, increased receptor sensitivity or perhaps a more complex interaction with other mediator pathways. Identifying biological and phenotypic characteristics related to CysLT effects and efficacy of LTRAs such as montelukast would allow for more defined clinical evaluations focused on subpopulations most likely to benefit from such treatments.

In previous studies, the relationship between the stable end-product of CysLT metabolism, leukotriene E₄ (LTE₄) and asthma control was examined (Rabinovitch N, Zhang L, Gelfand E W. Urine leukotriene E₄ levels were determined to be associated with decreased pulmonary function in children with persistent airway obstruction. J Allergy Clin Immunol 2006 September; 118 (3): 635-40).

While inhaled corticosteroids (ICS) are generally considered more potent than LTRAs and are therefore recommended as first-line therapy (Ducharme F M, Di Salvio F. Anti-Leukotriene agents compared to inhaled corticosteroids in the management of recurrent and/or chronic asthma in adults and children. The Cochrane Database of Systematic Reviews 2004, issue 1, Art. No. CD002314) treatment with ICS alone may not adequately control disease in 30% to 40% of patients (Heaney L G, Robinson D S. Severe asthma treatment: need for characterising patients. Lancet. 2005 Mar. 12-18; 3 65(9463): 974-6). This could reflect the inability of ICS therapy to reduce leukotriene production in certain subject subsets where this pathway predominates (Manso G, Baker A J, Taylor I K, Fuller R W. In vivo and in vitro effects of glucocorticosteroids on arachidonic acid metabolism and monocyte function in nonasthmatic humans. Eur Respir J 1992; 5:712-6; O'Shaughnessy K M, Wellings R, Gillies B, Fuller R W. Differential effects of fluticasone propionate on allergen-evoked bronchoconstriction and increased urinary leukotriene E4 excretion. Am Rev Respir Dis 1993; 147:1472-6). Although previous reports suggest that LABAs (long-acting beta-agonist) are superior to LTRAs in combination therapy (Ram F S F, Cates C J, Ducharme F M. Long acting beta-2 agonists versus anti-leukotrienes as add-on therapy to inhaled corticosteroids for chronic asthma. The Cochrane Database of Systematic Reviews 2005, Issue 1. Art. No. CD003137), other studies have reported that the addition of montelukast to ICS therapy is similar to LABAs in reducing symptoms and exacerbations of asthma (Ilowite J, Webb R, Friedman B, Kirwin E, Bird S R, Hustad C M, et al. Addition of montelukast or salmeterol to fluticasone for protection against asthma attacks: a randomized, double-blind, multicenter study. Ann Allergy Asthma Immunol 2004 June; 9 2(6): 641-8; Bjermer, L, Bisgaard H, Bousquet, J, Fabbri L M, Greening A P, Haahtela, T, et al. Montelukast and fluticasone compared with salmeterol and fluticasone in protecting against asthma exacerbation in adults: one year, double blind, randomized, comparative trial. BMJ2003; 327:891).

Individuals with asthma exposed to environmental agents such as second-hand smoke (SHS—also referred to as environmental tobacco smoke (ETS)), as well as individuals who smoke (primary tobacco exposure) may be at higher risk for severe exacerbations but biomarkers of susceptibility to environmental agent exposure have not been previously reported. Measurement of urinary leukotriene (uLTE₄) is a noninvasive method to assess changes in the rate of cysteinyl leukotriene (CysLT) production and excretion (Kumlin, M. Measurement of leukotrienes in humans. Am. J. Respir. Crit. Care Med. 2000; 161(suppl):S102-6; Smith C M, et al., Urinary leukotriene E4 in bronchial asthma. Eur. Respir. J. 1992; 5:693-699). Recent studies have found that leukotriene receptor antagonists are more effective in smokers (Rabinovitch, N., et al. Exposure to tobacco smoke increases leukotriene E4-related albuterol usage and response to montelukast. J. Allergy Clin. Immunol. 2008; 121:1365-71) or in children exposed to tobacco smoke (Rabinovitch, N., et al. Exposure to tobacco smoke increases leukotriene E4-related albuterol usage and response to montelukast. J. Allergy Clin. Immunol. 2008; 121:1365-71), suggesting that the CysLT pathway may play an important role in mediating asthma health effects related to SHS exposure.

Earlier studies have suggested that children exposed to significant SHS demonstrate an augmented response to leukotriene receptor antagonists such as monteleukast and a heightened response to unit increases in uLTE₄ (Rabinovitch N, et al. Exposure to tobacco smoke increases leukotriene E4-related albuterol usage and response to montelukast. J Allergy Clin Immunol 2008 June; 121(6): 1365-71) suggesting increased receptor sensitivity to CysLTs in the context of SHS exposure. Although it was previously assumed that CysLT effects occur predominantly through ligation of the CysLT₁ receptor by LTC₄, D₄ and less strongly by LTE₄, (Fregonese L, et al. Cysteinyl leukotrienes induce human eosinophil locomotion and adhesion molecule expression via a CysLT1 receptor-mediated mechanism. Clin Exp Allergy 2002; 32:745-750) recent studies suggest that LTE₄ alone may mediate multiple effects on asthma pathogenesis through P2Y (12), an adenosine diphosphate receptor, and the CysLT (E) receptor (Lee T H, Woszczek G, Farooque S P. Leukotriene E4: perspective on the forgotten mediator. J Allergy Clin Immunol 2009 September; 124(3):417-21; Austen K F, Maekawa A, Kanaoka Y, Boyce J A. The leukotriene E4 puzzle: finding the missing pieces and revealing the pathobiologic implications. J Allergy Clin Immunol 2009 September; 124(3):406-14).

Multiple studies have reported that children exposed to SHS are at risk for developing lower lung function, increased use of rescue medications, and more frequent hospitalizations. While multiple studies have reported that SHS is an important risk factor for increased asthma severity (Couriel, J M. Passive smoking and the health of children. Thorax. 1994; 49:731-734; Forastiere, F., et al. Effects of environment and passive smoking on the respiratory health of children. Int. J. Epidemiol. 1992; 21:66-73; Stoddard J J., et al. Impact of parental smoking on the prevalence of wheezing respiratory illness in children. Am. J. Epidemiol 1995; 141: 94-102; Chilmonczyk, B A., et al. Association between exposure to environmental tobacco smoke and exacerbations of asthma in children. N. Engl. J. Med. 1993; 328:1665-1669), there are few if any studies to date that have explored variables to help clinicians identify individuals at the highest risk for exacerbation thereby warranting more aggressive environmental or therapeutic interventions.

### SUMMARY OF INVENTION

The present invention provides for a method for predicting the risk for a subject of exacerbation of an inflammatory disease of the airways upon exposure to an environmental agent. The method includes determining the level of leukotriene selected from LTA₄, LTB₄, LTC₄, LTD₄, LTE₄, LTF₄ or combinations thereof in samples from the subject having exposure to or at risk of having exposure to the environmental agent. The presence of a variable leukotriene level in the samples from the subject identifies the subject as at being risk of exacerbation due to the inflammatory disease of the airways.

The present invention also provides for a method for reducing a subject's risk of exacerbation of an inflammatory disease of the airways upon exposure to an environmental agent. The method includes identifying a subject having exposure to or at risk of having exposure to the environmental agent as being at risk of exacerbation of an inflammatory disease of the airways by determining the level of leukotriene selected from LTA₄, LTB₄, LTC₄, LTD₄, LTE₄, LTF₄ or combinations thereof in samples from the subject. The presence of a variable leukotriene level in the samples from the subject identifies the subject as being at risk of exacerbation of the inflammatory disease of the airways. The method further includes treating the subject for the inflammatory disease of the airways.

The present invention further includes a method for predicting the risk for a subject of exacerbation of an inflammatory disease of the airways upon exposure to an environmental agent. The method includes determining the level of leukotriene selected from the group consisting of LTA₄, LTB₄, LTC₄, LTD₄, LTE₄, LTF₄ or combinations thereof in a sample from the subject having exposure to or at risk of having exposure to the environmental agent. The presence of an elevated leukotriene level in the sample from the subject identifies the subject as being at risk of exacerbation of the inflammatory disease of the airways.

In another embodiment, the present invention includes a method for reducing a subject's risk of exacerbation of an inflammatory disease of the airways upon exposure to an environmental agent. The method includes identifying a subject having exposure to or at risk of having exposure to the environmental agent as being at risk of exacerbation of an inflammatory disease by determining the level of leukotriene selected from LTA₄, LTB₄, LTC₄, LTD₄, LTE₄, LTF₄ or combinations thereof in a sample from the subject. The presence of an elevated leukotriene level in the sample identifies the subject as being at risk of exacerbation due to the inflammatory disease of the airways. The method further includes treating the subject for the inflammatory disease of the airways.

In some embodiments of the methods of the present invention, the methods further comprising determining whether the subject has exposure to or is at risk of having exposure to the environmental agent.

In some embodiments of the methods of the present invention, the step of determining the level of leukotriene in the sample comprises determining the level of LTE₄.

In some embodiments of the methods of the present invention, the inflammatory disease of the airways is asthma.

In some embodiments of the methods of the present invention, the inflammatory disease of the airways is airway hyper-responsiveness.

In some embodiments of the methods of the present invention, the inflammatory disease of the airways is triggered by the subject's exposure to an environmental agent. In some embodiments, the environmental agent is selected from second hand smoke (including environmental tobacco smoke), primary tobacco smoke, exhaust particles, gases, ozone or an allergen. In some embodiments, the subject's exposure to second hand smoke or primary tobacco smoke is determined by the subject's cotinine levels. In yet other embodiments, the allergen is selected from pollen and animal allergens. In still further embodiments, the inflammatory disease of the airways is triggered by the subject's exposure to second hand smoke or primary tobacco smoke.

In some embodiments of the methods of the present invention, the sample is a biological fluid. In yet other embodiments, the biological fluid is selected from urine, blood, sputum, saliva, exhaled breath condensate or bronchalveolar fluid.

In some embodiments of the methods of the present invention, the subject is being administered an inhaled corticosteroid.

Some embodiments of the present invention further comprise determining the subject's FENO level, and determining the ratio between the subject's LTE₄ level and the subject's FENO level wherein a high LTE₄:FENO ratio identifies the subject as at risk of exacerbation. In still a further embodiment, the high LTE₄:FENO ratio is at or above 4.0 ((pg/mg)/ppb).

In various embodiments the subject is human and in yet further embodiments the subject is a child.

In further embodiments of the method of the present invention the step of treating the subject for the inflammatory disease of the airways is selected from monitoring for exposure to the environmental agent; reducing exposure to the environmental agent; administering medication to treat the disease; or combinations thereof.

### BRIEF DESCRIPTION OF THE DRAWING

FIG. 1 shows a receiver operator characteristic curve showing the logistic regression model for the association of logLTE₄ and emergency department (ED) or urgent care (UC) visits in children exposed to significant SHS as determined by urinary cotinine levels. Specificity and sensitivity were 100% and 67% respectively at an uLTE₄ level at or about 106 pg·mg (Area under the curve=0.85, p=0.003).

### DETAILED DESCRIPTION

The present invention is directed toward methods for predicting the risk for a subject of exacerbation of an inflammatory disease of the airways upon exposure to an environmental agent. The invention includes determining the leukotriene level in a sample from the subject having been exposed to or at risk of having exposure to the environmental agent, wherein the presence of a variable leukotriene level in repeated samples from the subject identifies the subject as being at risk of exacerbation of the inflammatory disease of the airways. In another embodiment, the invention includes determining the leukotriene level in a sample from the subject having been exposed to or at risk of having exposure to the environmental agent, wherein the presence of an elevated leukotriene level in a sample from the subject having been exposed to or at risk of having exposure to the environmental agent identifies the subject as at risk of exacerbation of the inflammatory disease of the airways.

The present invention is also directed toward methods for reducing a subject's risk of exacerbation of an inflammatory disease of the airways upon exposure to an environmental agent. The invention includes determining the leukotriene level in a sample from the subject having been exposed to or at risk of having exposure to the environmental agent, wherein the presence of a variable leukotrine level in the sample from the subject identifies the subject as at risk of exacerbation of the inflammatory disease of the airways and treating the subject for the inflammatory disease of the airways, such as by monitoring for exposure to the environmental agent, reducing exposure to the environmental agent or administering medication to treat the disease. In another embodiment, the invention includes determining the leukotriene level in a sample from the subject having been exposed to or at risk of having exposure to the environmental agent, wherein the presence of an elevated leukotrine level in the sample from the subject identifies the subject as at risk of exacerbation of the inflammatory disease of the airways and treating the subject for the inflammatory disease of the airways, such as by monitoring for exposure to the environmental agent, reducing exposure to the environmental agent or administering medication to treat the disease.

Exacerbation of an inflammatory disease of the airways generally refers to a worsening of a disease, such as an increase in the severity or frequency of symptoms of the disease, or a decrease in the responsiveness to treatments for the disease.

In the methods of the present invention, the subject may have, or be at risk of developing, an inflammatory disease, and, in particular, diseases associated with airway inflammation. For example, airway inflammation is commonly associated with allergic inflammation, and/or viral-induced inflammation. The subject may thus have, or be at risk of developing, a condition including, but not limited to, any chronic obstructive disease of the airways. Such conditions include, but are not limited to any one or more of the following: asthma, airway hyper-responsiveness, chronic obstructive pulmonary disease, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease, hyper-eosinophilic syndrome, rhinitis, sinusitis, exercise-induced asthma, pollution-induced asthma and parasitic lung disease. Airway inflammation associated with viral-induced inflammation can occur in a patient that has, or is at risk of developing, an infection by a virus including, but not limited to any one or more of the following: respiratory syncytial virus (RSV), parainfluenza virus (Ply), rhinovirus (RV) and adenovirus.

In the methods of the present invention, the inflammatory disease of the airways can be one which is or symptoms of which are triggered or caused by exposure to an environmental agent. In various embodiments, exposure to environmental agents, includes but is not limited to any one or more of the following: second hand smoke (including ETS), primary tobacco smoke, diesel exhaust particles, ozone and an allergen (e.g. pollen, pet dander, and animal dander). In an embodiment of the present invention, exposure to an environmental agent can be determined by determining the subject's cotinine levels from urine, hair, saliva or other bodily fluids. In a specific embodiment, the cotinine level is determined from urine. Cotinine levels can be determined by means of immunoassay (Muscat, J. E., et al. Racial differences in exposure and glucuronidation of the tobacco-specific carcinogen 4-(methylnitrosamino)-1-(3-pyridyl)-1-butanone (NNK). Cancer 2005; 103:1420-6) or mass spectrometry assay.

Embodiments of the invention include determination of the subject's leukotriene level. The leukotriene can be LTA₄, LTB₄, LTC₄, LTD₄, LTE₄, LTF₄ or combinations thereof. In a preferred embodiment, the leukotrine is LTE₄. The subject's leukotriene level can be determined in a number of different ways, including methods currently known in the art, such as by analyzing a sample from a subject for the presence of a leukotriene. Suitable sample types include but are not limited to biological fluids, including urine, blood, sputum, bronchoalveolar fluid, saliva and exhaled breath condensate. Typically, LTE₄ levels are measured in the urine after excretion by methods such as mass spectrometry, radioimmunoassays, and enzyme immunoassays, or in exhaled breath condensates. A detailed discussion of LTE₄ and its measurement may be found in Rabinovitch, Immunol. Allergy Clin. N. Am. 27:651-664 (2007), the contents of which are incorporated herein by reference in its entirety. In addition, other indicators for determining leukotriene levels include, but are not limited to, direct measurement of LTA₄, LTB₄, LTC₄, LTD₄, LTE₄, LTF₄ levels or combinations thereof, in bronchoalveolar fluid as well as other biological fluids including but not limited to sputum (including induced sputum), saliva, exhaled breath condensate and blood.

In an embodiment of the present invention, the presence of a variable leukotriene level in the samples identifies the subject as being at risk of exacerbation of the inflammatory disease of the airways. A variable leukotriene level generally refers to the level of a leukotriene in an individual varying at different times, such as at the time that two or more samples are taken from an individual. Such differences in levels can be determined by measuring standard deviations in repeated samples. For example, samples can be taken from a subject on three consecutive days, typically at about the same time each day, to evaluate variability. Other suitable testing schedules can be used as well and it will be recognized that the ability to evaluate variability in leukotriene levels is not limited to any single or set of specific testing schedules. For example, an alternative testing schedule includes taking eight samples from a subject at about the same time of day over a two week period. Others testing schedules are suitable as well, for example, taking between three and eight samples or more samples on a schedule of no more than one sample a day with taking a sample every day, every two days or every three days. More particularly, in specific embodiments of the present invention, one sample is taken from the subject on consecutive days, wherein the number of consecutive days can be about 2 days, 3 days, 4 days, 5 days, 6 days, 7 days, 8 days, 9 days, 10 days, 11 days, 12, days, 13 days or 14 days. In other aspects, the sample is taken at the same time of day. In another aspect, one sample is taken on two separate days within about a 12 month span, 6 month span, 5 month span, 4 month span, 3 month span, 2 month span, 1 month span, 14 day span, 10 day span, 8 day span, 5 day span or 3 day span of each other. In another aspect, one sample is taken on 3 separate days, 5 separate days, 10 separate days, 20 separate days, or 30 separate days, within about a 12 month span, 6 month span, 5 month span, 4 month span, 3 month span, 2 month span or 1 month span. In one embodiment, one sample is taken on two separate days within a about a 6 month span of each other.

In one embodiment of the present invention, the presence of a variable leukotriene level in the sample from the subject having exposure to or at risk of having exposure to the environmental agent as compared to previous leukotriene levels and/or subsequent leukotriene levels from the subject identifies the subject as at risk of exacerbation due to the inflammatory disease of the airways. The term “variable leukotriene level” refers to a leukotriene level that varies from another leukotriene level using statistical methods. In particular, the higher the standard deviation of the leukotriene level for a given subject, the greater the risk for exacerbation. The standard deviation can be about 20 pg leukotriene (uLTE4)/mg creatinine, about 25 pg leukotriene/mg creatinine, about 30 pg leukotriene/mg creatinine, about 40 pg leukotriene/mg creatinine, about 50 pg leukotriene/mg creatinine, about 100 pg leukotriene/mg creatinine, about 125 pg leukotriene/mg creatinine, about 150 pg leukotriene/mg creatinine, about 175 pg leukotriene/mg creatinine, about 200 pg leukotriene/mg creatinine, about 225 pg leukotriene/mg creatinine, about 250 pg leukotriene/mg creatinine, about 250 pg leukotriene/mg creatinine, about 275 pg leukotriene/mg creatinine, about 300 pg leukotriene/mg creatinine, about 325 pg leukotriene/mg creatinine, about 325 pg leukotriene/mg creatinine, about 350 pg leukotriene/mg creatinine, about 375 pg leukotriene/mg creatinine, about 400 pg leukotriene/mg creatinine or about 450 pg leukotriene/mg creatinine. In a preferred embodiment, the standard deviation is at or above 25 pg leukotriene/mg creatinine.

In another embodiment of the present invention, the presence of an elevated leukotriene level in a sample from the subject having exposure to or at risk of having exposure to the environmental agent identifies the subject as being at risk of exacerbation of an inflammatory disease of the airways. To determine the leukotriene level in a subject, the level can be sampled on any suitable schedule. For example, the average of two samples taken on separate days is predictive of the leukotriene levels in a subject for the next six months. That is, if 2 samples averaged in an individual are “high” compared to others, that is very predictive of a “high” level over the next 6 months. So, 2 samples every 6 months would be sufficient in determining the level above a cut-off point. A leukotriene level can be considered to be high or elevated by reference to a cutoff number. For example, a leukotriene (uLTE₄) value of greater than about 60 pg/mg, 65 pg/mg, 70 pg/mg, 75 pg/mg, 80 pg/mg, 85 pg/mg, 90 pg/mg, 95 pg/mg, 100 pg/mg, 105 pg/mg, 110 pg/mg, 115 pg/mg, 120 pg/mg, 125 pg/mg, 130 pg/mg, 135 pg/mg, 140 pg/mg, 145 pg/mg, 150 pg/mg, 155 pg/mg, 160 pg/mg, 165 pg/mg, 170 pg/mg, 175 pg/mg, or 180 pg/mg can be appropriate elevated levels. Alternatively, a high or elevated leukotriene level can be determined by reference to a level in a subject that is higher than a suitable baseline level. Baseline levels can be determined, for example, by reference to leukotriene levels of healthy individuals, individuals with controlled asthma or individuals with controlled asthma while taking controller medications such as inhaled corticosteroids.

With respect to one of such population groups, an elevated leukotriene level can be an individual with a leukotriene level that is more than about 10% greater than leukotriene level of the baseline population, more than about 15% greater, more than about 20% greater, more than about 25% greater, more than about 30% greater, more than about 35% greater, more than about 40% greater, more than about 45% greater, more than about 50% greater, more than about 55% greater, more than about 60% greater, more than about 65% greater, more than about 70% greater, more than about 75% greater, more than about 80% greater, more than about 85% greater, more than about 90% greater, more than about 95% greater or more than about 100% greater, Alternatively, a high or elevated leukotriene level can be determined by evaluating the percentile of a cohort in which a given individual is. A cohort can be defined by any number of relevant parameters, such as age, condition (asthmatic, treated or not), height, weight, etc. For example, an individual that is in about the upper 50^{th} percentile of leukotriene values, about the upper 55^{th} percentile, about the upper 60^{th} percentile, about the upper 65^{th} percentile, about the upper 70^{th} percentile, about the upper 75^{th} percentile, about the upper 80^{th} percentile, about the upper 85^{th} percentile, about the upper 90^{th} percentile, or about the upper 95^{th} percentile can be considered to have a high or elevated leukotriene level.

In various aspects of the present invention, subjects found to be at risk of exacerbation due to inflammatory disease of the airways, can be treated. For example, such treatment can include monitoring for exposure to environmental agents, reducing exposure to environmental agents, administering medication to treat the disease or combinations thereof. For example, exposure to tobacco smoke can be monitored by measuring cotinine levels. Other environmental agents can be monitored in ways suitable to the particular agent. Treatment can also include decreasing exposure to the environmental agent, such as by reducing or eliminating a subject's exposure to second hand smoke, environmental tobacco smoke, primary tobacco smoke, allergens, exhaust particles, gases, or ozone. In other aspects, a subject determined to be at risk can be treated with a suitable medication, such as a leukotriene modifier. A leukotriene modifier includes any agent that modifies or inhibits the inflammatory activities of leukotrienes.

Such modifications or inhibitions can take place at a variety of levels. For example, the inflammatory activity of a leukotriene can be modified or inhibited by modifying or inhibiting leukotriene metabolism. For example, leukotriene metabolism can be effectuated by inhibition of 5-lipoxygenase or inhibition of 5-lipoxygenase-activating protein (FLAP) (e.g., Zileuton also known as ZYFLO® Abbott Laboratories, Abbott Park, Ill.). The inflammatory activity of a leukotriene can alternatively be modified or inhibited by modifying or inhibiting biological functioning of leukotrienes. For example, inhibition of the biological functioning of leukotrienes can be effectuated by agents that antagonize the actions of leukotriene receptors, (also known as leukotriene receptor antagonists or LTRAs). LTRAs include but are not limited to montelukast, zafirlukast (e.g. ACCOLATE® AstraZeneca, Wilmington Del.) and pranlukast. The biological functioning of leukotrienes can also be inhibited by interference with receptor binding such as by an antibody to a leukotriene. In another aspect, an increase in inhaled steroid doses is administered to the subject.

In another embodiment of the present invention includes determination of the subject's FENO, which can determine a subject's eosinophilic airway inflammation level. FENO levels in a subject may be measured or determined by any means known in the art. Typically, FENO levels are measured or determined in exhaled breath using nitric oxide analyzers, including but not limited to, chemiluminescence analyzers and electrochemical monitors, such as NIOX® (Aerocrine, Stockholm, Sweden), NIOX MINO® (Aerocrine, Stockholm, Sweden), SIEVERS® NO analyzer (Ionics Instrument, Boulder Colo.), CLD 88sp FENO analyzer (Eco Medics, Duernten, Switzerland), and LR2000 analyzer (Logan Research Ltd, Rochester, UK). A detailed discussion of FENO and its measurement may be found in Kharitonov et al., Chest 130:1541-1546 (2006), the contents of which are incorporated herein by reference in its entirety.

Preferably, FENO levels can be determined by exhaled breath using a nitric oxide analyzer, and are less than about 500 ppb, less than about 475 ppb, less than about 450 ppb, less than about 425 ppb, less than about 400 ppb, less than about 375 ppb, less than about 350 ppb, less than about 325 ppb, less than about 300 ppb, less than about 275 ppb, less than about 250 ppb, less than about 225 ppb, less than about 200 ppb, less than about 175 ppb, less than about 150 ppb, less than about 125 ppb, less than about 100 ppb, less than about 95 ppb, less than about 90 ppb, less than about 85 ppb, less than about 80 ppb, less than about 75 ppb, less than about 70 ppb, less than about 65 ppb, less than about 60 ppb, less than about 55 ppb, less than about 50 ppb, less than about 45 ppb, less than about 40 ppb, less than about 35 ppb, less than about 30 ppb, less than 29 ppb, less than 28 ppb, less than 27 ppb, less than 26 ppb, less than 25 ppb, less than 24 ppb, less than 23 ppb, less than 22 ppb, less than 21 ppb, less than 20 ppb, less than 19 ppb, less than 18 ppb, less than 17 ppb, less than 16 ppb, less than 15 ppb, less than 14 ppb, less than 13 ppb, less than 12 ppb, less than 11 ppb, less than 10 ppb, less than 9 ppb, less than 8 ppb, less than 7 ppb, less than 6 ppb, less than 5 ppb, less than 4 ppb, less than 3 ppb, less than 2 ppb or less than 1 ppb.

This embodiment of the present invention includes determining the ratio of a subject's FENO level and uLTE₄ level, wherein a high LTE₄:FENO ratio identifies the subject as at risk of exacerbation. For example, an LTE₄:FENO ratio at or above 4.0 (pg/mg)/ppb, 4.1 (pg/mg)/ppb, 4.2 (pg/mg)/ppb, 4.3 (pg/mg)/ppb, 4.4 (pg/mg)/ppb, 4.5 (pg/mg)/ppb, 4.6 (pg/mg)/ppb, 4.7 (pg/mg)/ppb, 4.8 (pg/mg)/ppb, 4.9 (pg/mg)/ppb, 5.0 (pg/mg)/ppb, 5.5 (pg/mg)/ppb, 6.0 (pg/mg)/ppb, 6.5 (pg/mg)/ppb, 7.0 (pg/mg)/ppb, 7.5 (pg/mg)/ppb, or 8.0 (pg/mg)/ppb is considered to be high and identifies the subject as at risk of exacerbation.

The methods of the present invention can be used in any animal subject, and particularly, in any vertebrate mammal, including, but not limited to, primates, rodents, livestock and domestic pets. Preferred mammals for the methods of the present invention include humans and even more preferably, children. Children are generally defined as having an age below the age of 18 years old.

As provided in the examples below, an increased risk of severe exacerbation in children with asthma who are exposed to environmental agents is shown. The relative risk of exacerbation was increased over 3.5 times in children living with parents who smoked or who demonstrated high mean cotinine levels and approximately 45% of children with SHS exposure experienced at least 1 asthma exacerbation requiring an emergency department (ED) or urgent care (UC) visit despite being in a school setting where asthma status was closely monitored and controller medications such as ICS were administered. A subject requiring a visit to the ED or UC can be considered to have severe asthma exacerbation.

The lack of generally accepted measurements for predicting increased individual risk for exacerbations in these children exposed to environmental agents is noteworthy. Neither symptom frequency, compromised lung function, airway inflammation as measured by FENO or even SHS dose as measured by mean urinary cotinine were related to exacerbations. Surprisingly, in contrast, uLTE₄ was shown to be a strong predictor of asthma exacerbations in children exposed to SHS with the odds of exacerbation increasing 14.6 times per unit increase in log uLTE₄. In these children, uLTE₄ levels at a clinically significant cut-off point (106 pg/mg) provided strong positive (100%) and negative (78%) predictive values identifying children who would require ED or UC visits due to exacerbation.

Besides providing insight into the mechanism of environmental agent-related asthma in children, the methods of the present invention demonstrate that LTE₄ is useful as a biomarker of asthma susceptibility in subjects (in particular children) exposed to environmental agents such as SHS. A child with asthma who is exposed to an environmental agent and has high or variable uLTE₄ levels, a more aggressive approach to minimizing exposure or sensitivity may be warranted. In this context, high or variable uLTE₄ levels would indicate sensitivity to, for example, SHS (analogous to a positive skin test) and SHS exposure would be determined by history and cotinine measurements. A determination of individual sensitivity to SHS along with SHS exposure would indicate the need for aggressive environmental interventions and/or specific medications in order to limit exposure, decrease sensitivity and prevent asthma exacerbations.

There is significant disease heterogeneity in asthma. As demonstrated in the examples below, subjects on controller medication who continue to exhibit persistent asthma symptoms may be separated into at least 2 heterogeneous groups based on their predominant sensitivities and exposures. Subjects not exposed to SHS may be more likely to report allergic symptoms in addition to their significant asthma. With schoolchildren, older age, female gender and lower percent predicted FEV₁ were significant predictors of severe exacerbations. An increased odds ratio of 3.32 per unit increase in log FENO levels was observed in the non-SHS exposed group although this association did not reach statistical significance. However, in children not exposed to significant SHS, a high LTE₄:FENO ratio of 4.8 was significantly associated with exacerbation risk providing an area under the curve of 0.87 for predicting the risk of asthma exacerbation. Other studies in schoolchildren with mild to moderate asthma have reported that FENO levels are correlated with classical inflammatory markers related to the allergic response such as peripheral blood eosinophils immunoglobulin E and serum eosinophil cationic protein while weakly correlated with levels of uLTE₄ indicating that CysLTs may be elevated independent of conventional steroid responsive eosinophilic inflammation measured by FENO (Strunk R C, et al for the Childhood Asthma Research and Education Network of the National Heart, Lung, and Blood Institute. Relationship of exhaled nitric oxide to clinical and inflammatory markers of persistent asthma in children. J Allergy Clin Immunol 2003; 112 (5):883-92). Subsequent studies have demonstrated that steroid-naïve children with high FENO levels respond preferentially to ICS (Szefler S J, et al. Characterization of within-subject responses to fluticasone and montelukast in childhood asthma. J Allergy Clin Immunol 2005; 115:233-42; Zeiger R S, et al.; Childhood Asthma Research and Education Network of the National Heart, Lung, and Blood Institute. Response profiles to fluticasone and montelukast in mild-to-moderate persistent childhood asthma. J Allergy Clin Immunol 2006; 117(1):45-52; Sorkness C A, et al. for the Childhood Asthma Research and Education Network of the National Heart, Lung, and Blood Institute. Long-term comparison of 3 controller regimens for mild-moderate persistent childhood asthma: the Pediatric Asthma Controller Trial. J Allergy Clin Immunol 2007; 119(1):64-72; Knuffman J E, et al. for the Childhood Asthma Research and Education Network of the National Heart, Lung, and Blood Institute. Phenotypic predictors of long-term response to inhaled corticosteroid and leukotriene modifier therapies in pediatric asthma. J Allergy Clin Immunol 2009; 123(2):411-6) and those with high uLTE₄ to FENO levels respond preferentially to LTRAs (Rabinovitch N, et al. for the Childhood Asthma Research and Education Network of the National Heart, Lung, and Blood Institute. Urinary leukotriene E4/exhaled nitric oxide ratio and montelukast response in childhood asthma. J Allergy Clin Immunol 2010 September; 126(3):545-51). The current examples demonstrate that these biomarker profiles may also indicate a differential sensitivity to specific exposures as children with high uLTE₄ levels were shown to be highly susceptible to SHS exposures, while those with an elevated or high LTE₄:FENO ratio were shown to be susceptible primarily to allergic triggers.

The results presented in the examples below show that subjects, in particular children, exposed to environmental agents, such as SHS, were at higher risk for asthma exacerbation than children without significant exposure to environmental agents. Urinary LTE₄ levels identified children with SHS exposure who experienced an exacerbation of their asthma requiring ED or UC visits. A cut-off point for uLTE₄ of 106 pg/mg achieved high predictive values while other more conventional predictors such as nighttime symptom frequency, pre-bronchodilator FEV₁ and FENO levels alone were unable to predict susceptibility in these children. In addition, not only the level of LTE₄ but the variability in LTE₄ levels also determines risk of exacerbation. A high day to day LTE₄ variability results in higher risk of exacerbation. Measurement of LTE₄ (such as uLTE₄ levels) may serve as a clinically important predictor of increased susceptibility in subjects such as children with asthma exposed to SHS, thus allowing for early environmental and therapeutic interventions to minimize severe asthma exacerbations in these at-risk subjects.

The following examples are provided for illustrative purposes, and are not intended to limit the scope of the invention as claimed herein. Any variations which occur to the skilled artisan are intended to fall within the scope of the present invention. All references cited in the present application are incorporated by reference herein to the extent that there is no inconsistency with the present disclosure.

### EXAMPLES

### Example 1

This example provides an analysis to quantify the increased risk of severe exacerbations with SHS exposure in children receiving inhaled corticosteroids, to assess the value of uLTE₄ in distinguishing children who are at increased risk when exposed to SHS, and to identify a predictive cut-off point for uLTE₄. In this way, a proof-of-concept for the clinical application of uLTE₄ as a biomarker test of individual susceptibility to severe exacerbations related to SHS was determined.

### Methods:

Children (N=44) with physician-diagnosed asthma, 6 to 15 years of age, at the Kunsberg School located on the campus of National Jewish Health, were followed for a 5 and a half month period (Dec. 3, 2007 through Apr. 17, 2008) as part of a separate NIH protocol studying the mechanisms of SHS and air pollution exposure on asthma. Ethical and scientific approval for collection of this data was obtained from the National Jewish Health's Institutional Review Board before recruitment.

The Kunsberg School is a public elementary school designed to address the educational needs of children with significant asthma that interferes with regular school attendance and progress. Many of the children are classified as urban poor with approximately two-thirds of the students receiving Medicaid assistance. Most of the children receive their daily medication at schools supervised by school nurses, thus minimizing compliance issues.

Up to 8 urine samples per subject were collected on consecutive school days during the first half of the study (until Feb. 20, 2008). Urine was collected at approximately the same time each day (11:00 AM to 1:00 PM), frozen at minus 70 degrees Celsius and then batch assayed for LTE₄ by mass spectrometry as previously described (Armstrong M, et al. Leukotriene-E4 in human urine: Comparison of on-line purification and liquid chromatography-tandem mass spectrometry to affinity purification followed by enzyme immunoassay. J Chromatogr B Analyt Technol Biomed Life Sci 2009 Oct. 1; 877(27):3169-74). Cotinine levels were determined by immunoassay (Muscat J E, et al. Racial differences in exposure and glucuronidation of the tobacco-specific carcinogen 4-(methylnitrosamino)-1-(3-pyridyl)-1-butanone (NNK). Cancer 2005; 103:1420-1426). Urinary LTE₄ levels were reported in picograms (pg) and standardized per milligram (mg) of creatinine Urinary cotinine levels were reported in nanograms (ng) per milliliter and standardized per mg of creatinine.

Fractional exhaled nitric oxide (FENO) levels were measured at the same time as urine collection using a NIOX (Aerocrine AB, Solna, Sweden) FENO analyzer at standard settings (Buchvald F, et al. Measurements of exhaled nitric oxide in healthy subjects age 4 to 17 years. J Allergy Clin Immunol 2005 June; 115(6): 1130-6) with the average of the 3 reproducible exhalations recorded. Once during the study period, children performed pre- and post-albuterol (4 puffs) spirometry on an office spirometer (Jaeger, Hochberg Germany) after discontinuing short (4 hours previously) and long-acting (24 hours previously) bronchodilator treatment. Weekly questionnaires recorded emergency department (ED) or urgent care (UC) visits for asthma. This information was crosschecked with parents and school nurses. At study entry parents described demographic variables, their child's atopic and asthma severity history, as well as household smoking habits.

### Statistical Analysis

The primary outcome variable was the presence of 1 or more ED or UC visits during the study period. The relative risk of ED or UC visits in children with significant SHS exposure (either parents reporting smoking on baseline questionnaire or cotinine levels above the mean for smoking households) was determined by Fisher's exact test for categorical variables and by T-tests for continuous variables. In the subgroup of children living with and without SHS exposure, models were constructed by logistic regression with log uLTE₄ as the predictor and the presence or absence of ED or UC visits as the outcome (primary analysis). For comparison, other potential predictors were also tested separately (secondary analyses). Urinary LTE₄ cut-off levels with the highest accuracy (true positive+true negatives divided by false positives+false negatives) were determined by constructing receiver operator curves in the logistic regression models. Associations between uLTE₄ levels at this cut-off point and demographic, biomarker or asthma severity outcomes were determined by Fisher's exact test for categorical variables and by T-tests for continuous variables. Positive predictive values were calculated as true positives (true positives+false positives) and negative predictive values were calculated as true negatives (true negatives+false negatives). Statistical analyses were performed using JMP software (SAS, NC). Statistical significance was reported for p-values below 0.05.

### Results:

44 schoolchildren primarily with moderate to severe asthma receiving inhaled corticosteroids were followed for 5 months with repeated measurements of urine LTE₄ and monitoring of ED and UC visits. SHS exposure status was determined by pre-study questionnaires and repeated measurements of urinary cotinine during the study. Children with significant SHS exposures were less likely to report an allergic history (p=0.002). Three of 24 (12.5%) children without significant SHS exposure experienced a severe asthma exacerbation while 9 of 20(45%) of children with SHS exposure experienced a severe exacerbation requiring an ED or UC visit (relative risk=3.6, 95^{th} confidence interval (CI) 1.1-11.5, p=0.02). Urinary LTE₄ levels were significantly associated with increased exacerbation risk in children exposed to SHS achieving an area under the curve of 0.85(p=0.004). Other predictors such as pre-bronchodilator lung function and exhaled nitric oxide were not related to exacerbations in this group. Urinary LTE₄ levels above 106 pg/mg achieved 67% (6/9) sensitivity and 100% (11/11) specificity for predicting children with SHS exposure who required an ED or UC visit. A high urinary LTE4: FENO ratio was associated with exacerbation risk in children not exposed to significant SHS. Urinary LTE₄ to FENO ratios above 4.8 achieved 100% (3/3) sensitivity and 67% (14/21) specificity for predicting children with SHS exposure who required an ED or UC visit.

TABLE 1
Demographic, asthma severity and biomarker characteristics for participants

| | | SHS Exposed | | |
|---|---|---|---|---|
| | | (household smoker or cotinine | | |
| Baseline | All Subjects | level > ln 3.1 ng/mg) | Not SHS Exposed | P-value for Difference |
| Characteristic | (N = 44) | (N = 20) | (N = 24) | SHS Vs no-SHS Exposed * |
| Age, years | 10.1 ± 2.3 | 10.4 ± 4.2 | 9.8 ± 2.0 | 0.23 |
| Height, inches | 55 ± 5.9 | 55.9 ± 6.5 | 54.2 ± 5.4 | 0.17 |
| Male, n (%) | 28 (64%) | 13 (65%) | 15 (62.5%) | 1.0 |
| African -American, n (%) | 29 (66%) | 14 (70.0%) | 15 (62.5%) | 0.75 |
| Allergies, no. (%) | 37 (84%) | 13 (65%) | 24 (100%) | 0.002 ** |
| Pre-bronchodilator | 97.1 ± 16.9 | 94.7 ± 15.2 | 99.1 ± 18.3 | 0.80 |
| FEV₁ (% predicted), | | | | |
| mean +/− SD | | | | |
| Pre-bronchodilator | 80.8 ± 9.6 | 80.2 ± 10.9 | 81.3 ± 8.5 | 0.64 |
| FEV₁/FVC ratio, | | | | |
| mean +/− SD | | | | |
| FEV₁ (% change with | 10.5 ± 7.7 | 10.7 ± 6.6 | 10.4 ± 8.9 | 0.44 |
| bronchodilator), | | | | |
| mean +/− SD | | | | |
| Nighttime symptoms > | 24 (54%) | 11 (55%) | 13 (54%) | 1.00 |
| 2x/week, no. (%) | | | | |
| Inhaled | 37 (84%) | 18 (90%) | 19 (79%) | 0.43 |
| corticosteroid, no. (%) | | | | |
| Cotinine (ng/mg | 35.0 ± 69.2 | 7.2 ± 90.2 | 3.9 ± 3.9 | <0.001 ** |
| [ln]), mean +/− SD | (2.1 ± 1.7) | (3.2 ± 1.4) | (0.9 ± 0.9) | |
| FENO (ppb [ln]), | 25.8 ± 23.2 | 27.2 ± 23.9 | 24.6 ± 23.0 | 0.31 |
| mean +/− SD | (2.9 ± 0.8) | (3.0 ± 0.8) | (2.9 ± 0.8) | |
| Urine LTE₄ (pg/mg | 90.9 ± 57.8 | 84.7 ± 58.1 | 96.1 ± 58.4 | 0.88 |
| creatinine) ([ln]), | (4.3 ± 0.6) | (4.2 ± 0.8) | (4.4 ± 0.5) | |
| mean +/− SD | | | | |

* P-values calculated using 2-tailed Fisher's Exact Test (for categorical variables) or Student's T-Test (for continuous variables).
** p < 0.01
FVC, Forced Vital Capacity

Sixty-four percent of the cohort (28/44) were male and 66% were African-American (at least 1 parent) with an average age of 10 years. Based on their baseline questionnaires, 15 out of 44 (34%) children lived with at least 1 parent who smoked and 11 (25%) lived with an indoor smoker. As questionnaire data tends to under-report smoking exposure (Cornelius M D, et al. Environmental tobacco smoke exposure in low-income 6-year-olds: parent report and urine cotinine measures. Nicotine Tob Res 2003; 3:333-339), cotinine data was used to identify additional children with significant SHS exposure. Five of the 29 remaining children (17%) were classified as SHS-exposed based on averaged log cotinine levels above the mean for children whose parents reported smoking (above log cotinine −1.46 mg/mg; log 3.1 ng/mg) so that 20 of 44 children were finally classified as SHS-exposed and 24 of 44 as non-exposed. None of the children were known smokers. Five children had cotinine/creatinine levels greater than 100. Three of these children had parents who reported smoking and two were regularly brought to school by relatives who smoked. Table 1 summarizes demographic, asthma severity, lung function and biomarker variables for the whole group (N=44) and stratified by children identified as exposed (N=20) or not exposed (N=24) to SHS. There were no significant differences observed between SHS exposed and non-exposed groups for age, height, gender, race, pre bronchodilator forced expiratory volume in 1 second (FEV₁), pre-bronchodilator FEV₁ to forced vital capacity (FVC) ratio, FEV₁ change (reversibility) with bronchodilator, nighttime symptom frequency, inhaled corticosteroid use, FENO uLTE₄ levels. Children classified as SHS exposed demonstrated significantly higher levels of urinary cotinine (p<0.001) and were less likely to report any allergic history of pet allergies, pollen, food allergies or eczema (p=0.002) (allergic). Three of the 24 (12.5%) non-SHS exposed children and 9 out of the 20 (45%) SHS exposed children required an ED or UC visit during the study period. None of the exacerbations occurred during the periods in which biomarker or lung function measurements were collected. Exposure to SHS was significantly (p=0.02) associated with need for ED or UC visit with a relative risk of 3.6 (95^{th} confidence interval 1.1-11.5).

TABLE 2
Associations with ED or UC visits in SHS and non-SHS Exposed Children

| | SHS Exposed | | Not SHS Exposed | |
|---|---|---|---|---|
| | Odds ratio per category or | | Odds ratio per category or | |
| Variable | unit increase in variable | p-value * | unit increase in variable | p-value * |
| Age (years) | 0.96 | 0.84 | 2.63 | 0.01 ** |
| Height (inches) | 0.98 | 0.82 | 1.26 | 0.06 |
| Male | 2.92 | 0.27 | Could not be | |
| | | | calculated**** | |
| African -American | 0.28 | 0.20 | 0.25 | 0.27 |
| Allergies | 1.14 | 0.89 | All allergic | |
| Pre-bronchodilator | 0.97 | 0.31 | 0.91 | 0.02*** |
| FEV₁ (% predicted) | | | | |
| Pre-bronchodilator | 1.005 | 0.89 | 0.92 | 0.25 |
| FEV₁/FVC (%) | | | | |
| FEV₁ change with | 0.92 | 0.27 | 1.11 | 0.17 |
| bronchodilator (% predicted) | | | | |
| Nighttime symptoms > | 1.04 | 0.96 | 1.82 | 0.64 |
| 2x/week | | | | |
| Inhaled | Could not be | | 0.47 | 0.59 |
| corticosteroid use | calculated^{#} | | | |
| Cotinine (mg/mg | 1.001 | 0.71 | 1.15 | 0.30 |
| creatinine) | | | | |
| FENO (ppb) | 1.01 | 0.61 | 1.03 | 0.17 |
| Urinary LTE₄ | 1.04 | 0.003*** | 1.00 | 0.81 |
| (pg/mg creatinine) | | | | |

* P-values calculated using likelihood ratio test
** p < 0.05
***p < 0.01
****all children with exacerbations were female
^{#}all children with exacerbations were receiving daily ICS therapy

Urinary LTE₄ levels in SHS exposed children were significantly (odds ratio (OR) 1.04 per unit increase in ul LTE4, p=0.003) associated with ED or UC visits such that children with higher log uLTE₄ levels were more susceptible than those with lower levels (Table 2). None of the other predictors tested were significantly associated with increased risk in SHS exposed children and mean uLTE₄ levels were not associated (p=0.81) with exacerbations in children not exposed to SHS (Table 2). Five out of 24 (21%) children in the non-SHS exposed group had uLTE₄ levels at or above 106 pg/mg. One of 3 children (33% sensitivity) with exacerbations in the non-exposed group had high uLTE₄ levels, and 17 of 21 children without exacerbations (81% specificity) had low uLTE₄ levels. Lower percent predicted FEV₁ (p=0.02), older age (p=0.01) and the uLTE4:FENO ratio (p=0.05) were associated with exacerbations in non-SHS exposed children but not in children exposed to SHS (Table 2). Gender was a significant variable (p=0.04) in the non-SHS group but since all exacerbations occurred in females, an odds ratio could not be calculated. All children in the non-SHS group reported allergies, thereby making associations not interpretable for allergic status in this group.

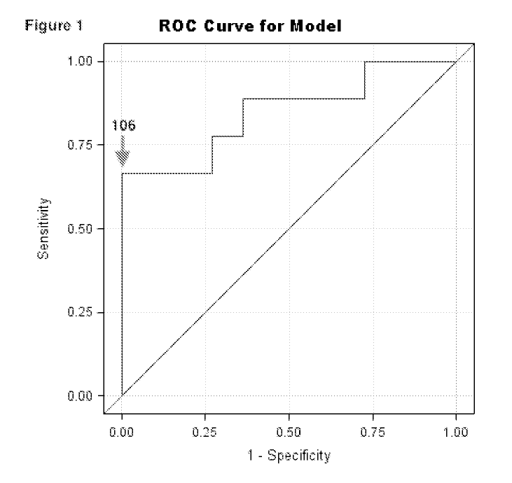

Receiver operator curves identified the most accurate cutoff point for uLTE₄ as at or above 106 pg/mg (FIG. 1). Six (30%) of the SHS-exposed children had mean LTE₄ levels above and 14 (70%) at or below this cutoff. Six of the nine SHS-exposed children who required ED or UC visits had mean uLTE₄ levels at or above 106 pg/mg (67% sensitivity) and 11/11 children who did not require an ED or UC visit had uLTE₄ levels below 106 pg/mg (100% specificity) (area under the curve 0.85, p=0.003) (FIG. 1).

Similar sensitivities, specificities and cut-off points for uLTE₄ and ED or UC visits were observed if the SHS-exposed group was classified by reported smoking alone or by cotinine levels (at or above 5 or 10 mg/mg) alone. Based on the prevalence of exacerbations in SHS-exposed children in this cohort (45%), the positive predictive value of the uLTE₄ test was 100% while the negative predictive value was 78%. Urinary LTE₄ levels above 106 pg/mg in SHS exposed children were not associated with age (p=0.26), height (p=0.29), gender (p=1.00), African-American race (p=1.00), allergic status (p=1.00), pre-bronchodilator FEV₁ percent predicted (p=0.77) or FEV₁/FVC ratio (p=0.77), FEV₁ percent predicted change post-bronchodilator (p=0.41), nighttime symptom frequency (p=1.00), ICS usage (p=1.00), log cotinine levels (p=0.43) or log FENO (0.11). A high urinary LTE4: FENO ratio was associated with exacerbation risk in children not exposed to significant SHS. Urinary LTE₄ to FENO ratios above 4.8 achieved 100% (3/3) sensitivity and 67% (14/21) specificity for predicting children with SHS exposure who required an ED or UC visit.

### Example 2

This example demonstrates variable leukotriene levels as a biomarker for exacerbation. Subjects with high day-to-day LTE₄ variable levels were determined to be at a higher risk of exacerbations. Up to 8 samples on consecutive school days were measured. Children exposed to SHS with high LTE4 variability (i.e. high standard deviation of LTE4) measurements were at high risk of exacerbations. Urinary LTE4 standard deviations at or above 25 pg/mg produced 78% (7/9) sensitivity and 100% (11/11) specificity.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

1. A method for predicting the risk for a subject of exacerbation of an inflammatory disease of the airways upon exposure to an environmental agent, comprising:
determining the level of leukotriene selected from the group consisting of LTA₄, LTB₄, LTC₄, LTD₄, LTE₄, LTF₄, and combinations thereof in samples from a subject having exposure to or at risk of having exposure to the environmental agent; and
wherein the presence of a variable leukotriene level in the samples identifies the subject as being at risk of exacerbation of the inflammatory disease of the airways.

2. A method for reducing a subject's risk of exacerbation of an inflammatory disease of the airways upon exposure to an environmental agent, comprising:
1. identifying a subject having exposure to or at risk of having exposure to the environmental agent as being at risk of exacerbation of an inflammatory disease of the airways by determining the level of leukotriene selected from the group consisting of LTA₄, LTB₄, LTC₄, LTD₄, LTE₄, LTF₄, and combinations thereof in samples from the subject;
wherein the presence of a variable leukotriene level in the samples identifies the subject as being at risk of exacerbation of the inflammatory disease of the airways; and
2. treating the subject for the inflammatory disease of the airways.

3. A method for predicting the risk for a subject of exacerbation of an inflammatory disease of the airways upon exposure to an environmental agent, comprising:
determining the level of leukotriene selected from the group consisting of LTA₄, LTB₄, LTC₄, LTD₄, LTE₄, LTF₄, and combinations thereof in a sample from a subject having exposure to or at risk of having exposure to the environmental agent; and
wherein the presence of an elevated leukotriene level in the sample identifies the subject as being at risk of exacerbation of the inflammatory disease of the airways.

4. A method for reducing a subject's risk of exacerbation of an inflammatory disease of the airways upon exposure to an environmental agent, comprising:
1. identifying a subject having exposure to or at risk of having exposure to the environmental agent as being at risk of exacerbation of an inflammatory disease of the airways by determining the level of leukotriene selected from the group consisting of LTA₄, LTB₄, LTC₄, LTD₄, LTE₄, LTF₄, and combinations thereof in a sample from the subject;
wherein the presence of an elevated leukotriene level in the sample identifies the subject as being at risk of exacerbation due to the inflammatory disease of the airways; and
2. treating the subject for the inflammatory disease of the airways.

5. The method of claim 1 further comprising determining whether the subject has exposure to or is at risk of having exposure to the environmental agent.

6. The method of claim 1, wherein the step of determining the level of leukotriene in the sample comprises determining the level of LTE₄.

7. The method of claim 1, wherein the inflammatory disease of the airways is asthma.

8. The method of claim 1, wherein the inflammatory disease of the airways is airway hyper-responsiveness.

9. The method of claim 1, wherein the inflammatory disease of the airways is triggered by the subject's exposure to an environmental agent selected from the group consisting of second hand smoke, primary tobacco smoke, exhaust particles, gases, ozone and an allergen.

10. The method of claim 9, wherein the subject's exposure to second hand smoke is determined by the subject's cotinine levels.

11. The method of claim 9, wherein the allergen is selected from the group consisting of pollen and animal allergens.

12. The method of claim 1, wherein the inflammatory disease of the airways is triggered by the subject's exposure to second hand smoke.

13. The method of claim 1, wherein the inflammatory disease of the airways is triggered by the subject's exposure to primary tobacco smoke.

14. The method of claim 1, wherein the sample is a biological fluid selected from the group consisting of urine, blood, sputum, salvia, exhaled breath condensate and bronchoalveolar fluid.

15. The method of claim 1, wherein the subject is being administered an inhaled corticosteroid.

16. The method of claim 1 further comprising determining the subject's fractional exhaled nitric oxide (FENO) level, and determining the ratio between the subject's LTE₄ level and the subject's FENO level wherein a high LTE₄:FENO ratio identifies the subject as at risk of exacerbation.

17. The method of claim 16, wherein the LTE₄:FENO ratio is at or above 4.0 ((pg/mg)/ppb).

18. The method of claim 1, wherein the subject is human.

19. The method of claim 18, wherein the subject is a child.

20. The method of claim 2, wherein the step of treating the subject for the inflammatory disease of the airways is selected from the group consisting of monitoring for exposure to the environmental agent; reducing exposure to the environmental agent; administering medication to treat the disease; and combinations thereof.
